(19) United States
(12) Patent Application Publication
(10) Pub. No.: US 2014/0234381 A1
(43) Pub. Date: Aug. 21, 2014

(54) SCAFFOLD FOR SUBRETINAL CELL TRANSPLANTATION AND DRUG DELIVERY

(71) Applicants: The Charles Stark Draper Laboratory, Inc., Cambridge, MA (US); The Schepens Eye Research Institute, Inc., Boston, MA (US)

(72) Inventors: Sarah L. Tao, Cambridge, MA (US); Stephen Redenti, New York, NY (US); Magali Saint-Geniez, Somerville, MA (US); Michael Young, Gloucester, MA (US); Patricia D'Amore, Boston, MA (US)

(73) Assignees: The Charles Stark Draper Laboratory, Inc., Cambridge, MA (US); The Schepens Eye Research Institute, Inc., Boston, MA (US)

(21) Appl. No.: 14/138,902

(22) Filed: Dec. 23, 2013

Related U.S. Application Data
(63) Continuation of application No. PCT/US2012/043692, filed on Jun. 22, 2012.
(60) Provisional application No. 61/499,909, filed on Jun. 22, 2011.

(51) Int. Cl. A61L 27/36; A61L 27/54

(52) U.S. Cl. CPC A61L 27/3604; A61L 27/54
USPC 424/400; 424/93.7; 435/396; 435/366; 264/41

(57) ABSTRACT

The following disclosure provides compositions and methods for the repairing of a diseased or disordered retina, for example, in patients suffering from age-related macular degeneration (AMD).

### CROSS-REFERENCE TO RELATED APPLICATIONS

This application claims the benefit of U.S. Provisional Application 61/499,909 filed Jun. 22, 2011, which is incorporated herein by reference in its entirety.

### BACKGROUND

In the U.S., age-related macular degeneration (AMD) is the leading cause of blindness among the elderly. The major causes of severe visual loss among patients with AMD are: (1) choroidal new vessel (CNV) growth under the fovea, and (2) atrophy of the fovea and subfoveal retinal pigment epithelium (RPE) and choroid. Currently, there is no treatment for atrophy, and although pharmacological therapy for CNVs can be effective, it results in significant visual improvement in only 25-40% of patients. During CNV, leakage of newly formed capillaries occurs behind the retina and damages the RPE and consequently photoreceptor cells, leading to loss of central vision.

The hallmark for these ocular diseases is the degeneration and loss of RPE cells. RPE cells are critical for the survival and proper function of the photoreceptors. In AMD, pathological changes are not limited to the RPE, but also affect the structure of the Bruch's membrane (BrM). BrM is a thick elastic lamina (1-4 pm thick, depending on the retinal region) that is interposed between the basal surface of the RPE and its blood supply, the choriocapillaris. Previous studies have suggested that effective strategies for RPE replacement will include the restoration of the subretinal matrix architecture that supports cell graft survival and function. For example, it has been seen that after macular translocation, vision in AMD eyes can be improved by providing relatively healthy RPE-Bruch's membrane-choroid complex under the fovea.

RPE tissue transplants have been shown to rescue photoreceptors and preserve visual acuity in animal models of retinal degeneration, but tissue transplantation in patients with RP or AMD (atrophic and neovascular) typically has produced limited visual recovery regardless of the type of tissue transplanted (e.g., autologous or allogeneic, adult or fetal). Potential causes of transplant failure in human patients include immune rejection and inflammation, inability of transplanted cells to survive and differentiate, and choriocapillaris atrophy, all causing graft death.

In addition, an important distinction between humans with AMD and laboratory animals is the age-related modification of Bruch's membrane that occurs in human eyes. This modification of Bruch's membrane may have a significant effect on RPE graft survival in patients with AMD. With normal aging, human Bruch's membrane, especially in the submacular region, undergoes numerous changes (e.g., increased thickness, deposition of extracellular matrix and lipids, cross-linking of protein, nonenzymatic formation of advanced glycation end products). It has been reported that an accelerated decline in the presence of laminin, fibronectin, and collagen IV in the RPE basement membrane with AMD. It is possible that changes in submacular Bruch's membrane permeability and choriocapillary density may contribute to age-related host and donor RPE death. Accordingly, for those patients experiencing severe central visual loss due to AMD, as well as those patients with retinal and/or RPE cell degeneration, there remains a need for reconstructing the RPE cells and forming a suitable interface with Bruch's membrane.

### SUMMARY

The present disclosure relates generally to the field of treatment of eye disorders, in particular retinal disorders such as age-related macular degeneration, retinitis pigmentosa, and other retinal diseases. One aspect of the invention relates to a biocompatible scaffold containing elements to provide 1) neuroprotection of photoreceptors, 2) reconstruction of the RPE, and 3) prevention of CNV. The invention provides methods and apparatus for growing and differentiating different sources of cells on biodegradable polymer substrates for the purpose of ocular transplantation, while providing controlled release of therapeutic molecules. The assembly serves as a basis for transplantation in patients undergoing both CNV excision and in patients with geographic atrophy.

One aspect of the disclosure relates to a composition comprising retinal pigment epithelium (RPE) cells adhering to a poly(ε-caprolactone) (PCL) membrane, wherein the PCL membrane comprises a plurality of pores distributed over the PCL membrane, each pore having a diameter of less than 1 micron. In some implementations, features of the pores are defined, for example, the pores may range in size from about 0.1-1 micron, or about from 300-500 nm. The pores may be substantially evenly distributed over the surface of the PCL membrane. The pores may be spaced at least about 1 micron from another pore.

In certain implementations, the PCL membrane has one or more specific physical characteristics. The PCL membrane may be flexible. The PCL membrane may have two surfaces that differ in texture from one another. For example, a first surface on the PCL membrane may be smooth, while a second surface of the PCL membrane comprises an anchoring structure. The anchoring structure may be a nanostructure or microstructure that adheres to an ocular tissue, such as Bruch's membrane. An exemplary anchoring structure comprises nanowires. In some implementations, the anchoring structure comprises a surface-modifying layer that enhances biocompatibility. This enhancement may be mediated by a surface modifying layer that comprises ECM molecules, and/or by a surface-modifying layer that induces apoptosis of inflammatory cells.

In some implementations, the PCL membrane is biodegradable, e.g., after implantation in the eye. The biodegradation may occur on a rapid scale or on a slow scale, for example, over the course of 1-5 years. In some implementations, the porous membrane comprises a polymer selected from poly(lactic-co-glycolic acid) (PLGA), poly(DL-lactide-co-ε-caprolactone) (DLPLCL), collagen, gelatin, agarose, poly(methyl methacrylate), gelatin/ε-caprolactone, collagen-GAG, collagen, fibrin, PLA, PGA, PLA-PGA co-polymers, poly(anhydrides), poly(hydroxy acids), poly(ortho esters), poly(propylfumarates), poly(caprolactones), poly(hydroxyvalerate), polyamides, polyamino acids, polyacetals, biodegradable polycyanoacrylates, biodegradable polyurethanes, polysaccharides, polypyrrole, polyanilines, polythiophene, polystyrene, polyesters, nonbiodegradable polyurethanes, polyureas, poly(ethylene vinyl acetate), polypropylene, polymethacrylate, polyethylene, polycarbonates, poly(ethylene oxide). Alternatively, the porous membrane may comprise a copolymer of PCL and at least one of the polymers listed above.

An exemplary feature of the PCL membrane is its release of therapeutic agents into a biologic environment, such as the eye. Therapeutic agents may induce apoptosis of inflammatory cells, suppress an immune response, reduce degeneration of retinal neurons, and/or inhibit angiogenesis. For example, the therapeutic agent may reduce activity of VEGF.

In some implementations, the RPE cells adhering to the membrane are mammalian cells. In alternative implementations, the RPE cells are not mammalian cells. The mammalian cells may be human cells, for example, fetal cells or adult cells. The mammalian cells may be immortalized human cells, such as ARPE-19 cells. In some implementations, the human cells are stem cells. Stem cells may be selected from embryonic, placental, umbilical, mesenchymal, progenitor, or induced pluripotent stem cells. In exemplary implementations, the stem cells are embryonic stem cells. In further implementations, the stem cells are induced pluripotent stem cells.

The RPE cells may form a monolayer of cells on the PCL membrane. In some implementations, the microvilli on the RPE cells are evenly distributed on the cell surface. The RPE cells may express genes characteristic of differentiated RPE. Exemplary genes may be selected from at least one of RPE65, Otx2, Tfeb, Na+K+ pump, CRALBP, PEDF, and VEGF. RPE cells may be phagocytic.

Another aspect of the disclosure relates to a method for treating symptoms of a retinal disorder in a patient, comprising implanting in one or both eyes of the patient the composition of any of the claims as filed herewith, or as described in the present application. Accordingly, a method for treating symptoms of a retinal disorder in a patient may comprise: (a) obtaining one or more stem cells from the patient or from a donor; (b) inducing the one or more stem cells to differentiate into retinal pigment epithelium (RPE) cells; (c) contacting the RPE cells with a porous poly(ε-caprolactone) (PCL) membrane, whereby the RPE cells adhere to the porous PCL membrane; and (d) implanting the porous PCL membrane and RPE cells from step (c) into one or both eyes of a patient suffering from a retinal disorder. The retinal disorder may be age-related macular degeneration or retinitis pigmentosa. Symptoms of a retinal disorder may comprise choroidal new vessel growth (CNV), atrophy of the fovea, atrophy of the subfoveal retinal pigment epithelium, atrophy of the choroid, and/or loss of central vision.

In some implementations, the porous PCL membrane comprises a plurality of pores, each pore having a diameter of less than 1 micron. The pores may range in size from 0.1-1 micron, for example, the pores may range in size from 300-500 nm. In some implementations, the pores are evenly distributed over the surface of the PCL membrane. A first surface of the porous PCL membrane may be smooth, and/or a second surface of the porous PCL membrane may comprise an anchoring structure. The PCL membrane may adhere to an ocular structure, for example, to Bruch's membrane. The porous PCL membrane may release one or more therapeutic agents after implantation. In the patient, the RPE cells growing on the porous PCL membrane may restore central vision.

Another aspect of the disclosure relates to a method for manufacturing a composition comprising retinal pigment epithelium (RPE) cells and a porous poly(8-caprolactone) (PCL) membrane, comprising contacting the RPE cells with a porous PCL membrane, whereby the RPE cells adhere to the porous PCL membrane. In some implementations, the method further comprises manufacturing the porous PCL membrane by sacrificial molding, injection molding, material printing, and/or laser machining.

In some implementations of the disclosure, the porous PCL membrane comprises a plurality of pores, each pore having a diameter of less than 1 micron. The pores may range in size from 0.1-1 micron, for example, the pores may range in size from 300-500 nm. The pores may be evenly distributed over the surface of the porous PCL membrane. In some implementations, a first surface on the PCL membrane is smooth, and/or a second surface on the porous PCL membrane comprises an anchoring mechanism. In some implementations, the porous PCL membrane releases a therapeutic agent into a biologic environment. In some implementations, the method for manufacturing a composition comprising RPE cells and a porous PCL membrane further comprises culturing the RPE cells on the porous PCL membrane and implanting the RPE cells and the porous PCL membrane into a patient. In some implementations, the RPE cells are non-human cells. In some implementations, the RPE cells are human cells. The human cells may be, for example, fetal cells or adult cells. The human cells may be immortalized human cells, such as ARPE-19 cells. In some implementations, the human cells are stem cells. Stem cells may be selected from embryonic, placental, umbilical, mesenchymal, progenitor, or induced pluripotent stem cells. In exemplary implementations, the stem cells are embryonic stem cells. In further implementations, the stem cells are induced pluripotent stem cells.

### BRIEF DESCRIPTION OF THE DRAWINGS

FIG. 1 shows adhesion of human retinal pigment epithelium cells to a poly(8-caprolactone) (PCL) membrane.

FIGS. 2A and B show rapid and sustained tight junction formation of human RPE cells on the PCL membrane.

FIG. 3A-D shows improved structural morphology of RPE cells cultured on the PCL membrane.

FIG. 4A-C shows gene expression analysis of cells from the immortalized RPE cell line APRE-19 cultured on a PCL membrane.

FIG. 5A-C shows phagocytic activity of ARPE-19 cells cultured on the PCL membrane.

FIG. 6A-D shows adhesion and differentiation of RPE cells derived from induced pluriopotent stem cells on the PCL membrane.

FIG. 7A-B shows a diagram of the differentiation protocol used to produce RPE cells from murine induced pluripotent stem cells (iPSCs).

FIG. 8 shows the release profile of FITC-albumin released from PCL in PBS over a three-week period.

FIG. 9 shows the number of living Human Umbilical Vein Endothelial Cells (HUVECs) as a function of Pigment Epithelium Derived Factor (PEDF) concentration (μg/mL).

FIG. 10 illustrates the effects of PEDF effect on HUVECs.

FIG. 11 demonstrates the effects of PEDF on photoreceptors.

FIG. 12A shows representative images of an exemplary photomask design and 12B shows RPE cells on the porous PCL scaffold stained with hematoxylin and eosin to demonstrate relative size. Arrowheads indicate several of pores.

FIG. 13 shows scanning electron microscopy images of A) an exemplary silicon scaffold, B) an exemplary PCL scaffold, C) the bottom of an exemplary PCL film after delamination, and D) the top of an exemplary PCL film after delamination.

FIG. 14A-F shows a diagram of an exemplary mold and scaffold fabrication process.

FIG. 15A-C shows costaining of ZO-1 (green) and DAPI (blue) on fetal human RPE cells cultured for 4 weeks on (A) polyester transwells, (B) non-porous PCL, and (C) porous PCL.

FIG. 16 shows transepithelial resistance of fetal human RPE on polyester transwells (white) and porous PCL (gray). ** indicates p<0.01, *** indicates p<0.001.

FIG. 17 shows RPE gene expression of the key visual product recycling proteins (A) RPE65 and (B) CRALBP at 1 and 4 weeks cultured on polyester transwells (gray), PCL (white), or porous PCL (black) at the mRNA level. * indicates p<0.05, ** indicates p<0.01, *** indicates p<0.001.

FIG. 18 shows RPE gene expression of the growth factors (A) PEDF and (B) VEGF cultured for 1 or 4 weeks on polyester transwells (gray), PCL (white), or porous PCL (black) at the mRNA level. * indicates p<0.05.

FIG. 19 shows RPE secretion of (A) PEDF and (B) VEGF cultured for 4 weeks on polyester transwells (PET), PCL, or porous PCL (POR). * indicates p<0.05, *** indicates p<0.001.

FIG. 20 shows phagocytic uptake of bovine outer segments by RPE cultured for 4 weeks on polyester transwells (PET), PCL, or porous PCL (POR) quantified using two methods of fluorescent quantification: (A) FITC pre-labeling and (B) rhodopsin post-labeling.

FIG. 21 shows the number of non-adhering cells per well with different surface modifications after 24 hours. Lower values indicates improved adhesion. * indicates p<0.05, ** indicates p<0.01.

FIG. 22 shows a fundus image of the PCL film implanted into the sub-retinal space of a C57B1/6J mouse. Arrow indicates implant location.

FIG. 23 shows optical coherence tomography (“OCT”) images of a C57B1/6J mouse retina (A) under normal conditions and (B) after sub-retinal saline injection (to create a pocket) and implantation of the PCL film.

### DETAILED DESCRIPTION

One aspect of the present invention relates to the reconstruction of retinal pigment epithelium (RPE) and a suitable interface with Bruch's membrane. The artificial Bruch's membrane comprises a polymer substrate, adapted to support growth, differentiation and/or maintenance of RPE cells on the top surface. The bottom surface is adapted to provide an anchoring mechanism to attach to the Bruch's membrane of a patient. Concurrently, the artificial membrane is designed to provide regularly spaced submicron pores for appropriate transport across the artificial substrate. The substrate may provide sustained release of molecules (such as Pigment Epithelium Derived Factor (PEDF), a factor which is biologically derived, endogenous to the retina, and synthesized by healthy RPE cells) for both neuroprotection and anti-angiogenesis. Accordingly, the composition described herein promotes growth, differentiation and/or maintenance of RPE. In some implementations, the composition blocks ingress of pathological neovascular structures, and provides a means to provide targeted delivery of therapeutic molecules subretinally at the site of action, avoiding the use of high dose drugs during intraocular injection or topical delivery.

In some implementations, the biological scaffold comprises nutrients to provide 1) neuroprotection of photoreceptors, 2) reconstruction of the RPE cells, and 3) reduce symptoms associated with macular degeneration. A variety of symptoms are associated with dry macular degeneration and/or with wet macular degeneration. Symptoms to be reduced include, but are not limited to: abnormal blood vessel growth in the choriocapillaries and through Bruch's membrane, leakage of blood and protein below the macula, scarring, damage to the photoreceptors, drusen, pigmentary alterations, hemorrhages of the eye, exudates, changes to the subretinal, sub-RPE, and/or intraretinal fluid, incipient atrophy, geographic atrophy, loss of visual acuity, blurred and/or distorted vision, central scotomas, loss of ability to discern colors, loss of recovery of visual function following exposure to bright lights, and a loss in contrast sensitivity.

Described herein are methods and apparatus for growing and/or differentiating different sources of cells on biodegradable or nondegradable polymer substrates for ocular implantation, optionally while providing controlled release of therapeutic molecules. In some implementations, the composition comprising RPE cells may serve as a basis for transplantation in patients undergoing both CNV excision and in patients with geographic atrophy. For example, the patients may be those experiencing severe central visual loss due to AMD, and/or patients experiencing retinal and/or RPE degeneration due to other causes.

### 1. Membrane

One aspect of the invention relates to a porous, thin-film membrane which may be used as a biological scaffold upon which RPE cells proliferate and/or differentiate. Numerous synthetic substrates for cell differentiation and transplantation have been developed, and polymer membranes that support RPE proliferation and differentiation in vitro include polyester, polyurethane, polycarbonate, and poly(DL-Lactic-co-glycolic acid) (PLGA). In addition, tissue engineering strategies have been used to culture RPE cells on chemically micropatterned PLGA scaffolds and on a human lens capsule, allowing the anchorage-dependent cells a supportive matrix to survive and grow, as well as on extracellular matrix produced by corneal cells.

In some implementations, the thin membrane may comprise a variety of nondegradable or degradable materials, including, for example, but not limited to, poly(lactic-co-glycolic acid) (PLGA), poly(DL-lactide-co-ε-caprolactone) (DLPLCL), poly(ε-caprolactone) (PCL), collagen, gelatin, agarose, poly(methyl methacrylate), gelatin/ε-caprolactone, collagen-GAG, collagen, fibrin, PLA, PGA, PLA-PGA co-polymers, poly(anhydrides), poly(hydroxy acids), poly(ortho esters), poly(propylfumarates), poly(caprolactones), poly(hydroxyvalerate), polyamides, polyamino acids, polyacetals, biodegradable polycyanoacrylates, biodegradable polyurethanes and polysaccharides, polypyrrole, polyanilines, polythiophene, polystyrene, polyesters, non-biodegradable polyurethanes, polyureas, poly(ethylene vinyl acetate), polypropylene, polymethacrylate, polyethylene, polycarbonates, poly(ethylene oxide), copolymers of the above, mixtures of the above, and adducts of the above, or combinations thereof. Thus, in some implementations, the thin membrane comprises a copolymer of PCL and at least one of the polymers listed above. Features of some exemplary porous membranes are described in U.S. 20110004304 (incorporated by reference herein in its entirety).

In some implementations, the thin membrane is a poly(ε-caprolactone) (PCL) membrane. As described in U.S. 20090306772 (incorporated by reference herein in its entirety), PCL is well-tolerated in the subretinal space in a large animal model (pig), and does not elicit an immune cell response and/or loss of integrity of the overlying retinal cytoarchitecture. Thus, RPE cells are able to grow on the PCL membrane, and overlying photoreceptor cells appear undisturbed despite their juxtaposition to the RPE-PCL composition. In addition, PCL may prevent differentiation of co-cultured progenitor cells, which means that PCL membranes may be used to graft undifferentiated cells or cells previously induced to differentiate along a cellular lineage. PCL membranes may also be modified to release factors that influence cellular differentiation, either before or after implantation into a host.

In some implementations, the membrane is flexible. The membrane may be biodegradable, and may degrade slowly over a span of months or years. The membrane may have a Young's modulus of at least 0.1 MPa to about 500 MPa and may have a thickness in a range from about 2 μm to about 6 μm. In some implementations, the membrane thickness may be from about 2 μm to about 25 μm, from about 5 μm to about 20 μm, or from about 10 μm to about 15 μM. In some implementations, the thickness may be up to 25 μm. The membrane may be characterized by a diffusivity in the range from about 200 μg/mm² per day to about 300 μg/mm² per day, for example, 250 μg/mm² per day, which is the estimated diffusivity of a native Bruch's membrane.

The membrane may have pores distributed over the surface. The pores allow diffusion of nutrients across the membrane and allow cells to communicate through extracellular signaling across the membrane, in order to support RPE proliferation and differentiation. At the same time, pores are preferably not large enough to allow cells to migrate or infiltrate through the membrane. Suitable pores may have diameters of less than about 1 μm, for example, the pores may be sized in range between about 0.1-1.0 μm. In some implementations, pores range in diameter from about 300-500 nm, for example, pores may be about 250, 275, 300, 325, 350, 375, 400, 425, 450, 475, or about 500 nm. In some implementations, pores are about 100, 200, 300, 400, 500, 600, 700, 800, 900 nm in diameter. In some implementations, pores are about 0.1 μm to 2.0 μm, from about 0.3 μm to about 1.7 μm, from about 0.5 μm to about 1.5 μm, or from about 0.8 μm to about 1.2 μm. In some implementations, the pores are about 0.8 to about 1.2 μm. All of the pores in the PCL membrane may be the same size, or the pores may have different sizes.

Uniformity in pore size and/or pore spacing may enable RPE cells to grow in a uniform layer on the membrane, and importantly, to form tight junctions with one another. One function of RPE cells is to regulate molecules that cross into the retina. In addition, tight junctions between RPE cells form a continuous barrier. Thus, in some implementations, pores are substantially evenly distributed over the surface of the membrane. The pores may be arrayed in a regular pattern. For example, pores may be spaced about 1.0 μm from other pores. In some implementations, pores may be spaced at least 1.0 μm from other pores. In some implementations, the pores are substantially round. As described in U.S. 20110004304 (incorporated by reference herein in its entirety), pores may alternately have a hexagonal shape, or a modified hexagonal shape wherein the straight edges of a hexagon are replace by curved convex or concave edges. Clusters of pores (e.g., round pores) may be also arranged hexagonally, for example, six or more round pores may be hexagonally arranged so that their overlap results in a modified hexagonal shape with convex edges. The larger pore sizes may be advantageous in that they enable multiple neighboring cells to directly interact, yet provide contact guidance for cell alignment along the grooves of the pores.

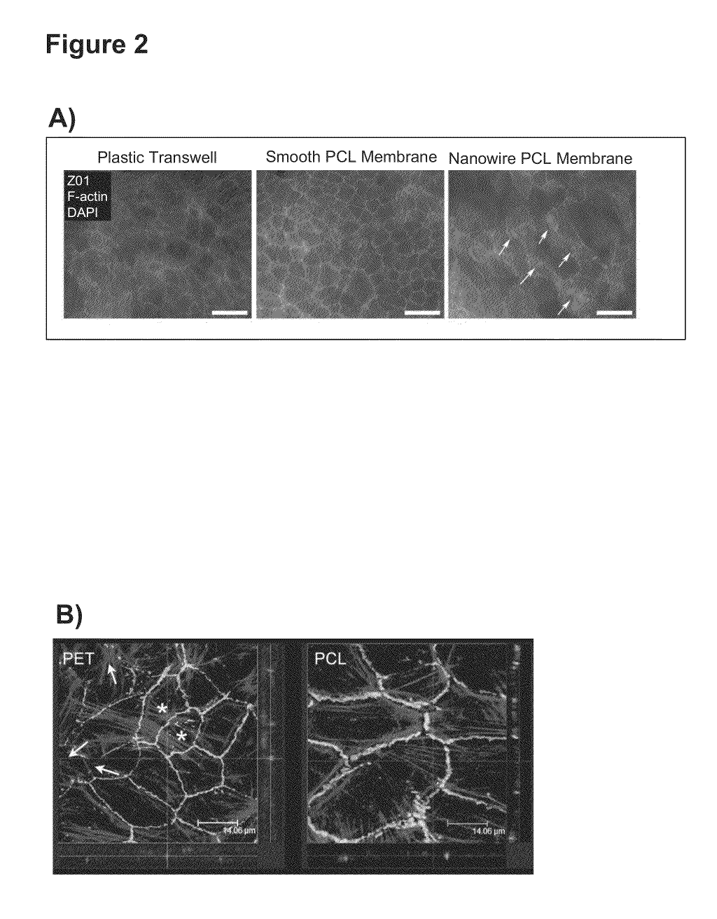

In certain implementations, a first surface of the membrane is smooth (i.e., has a no surface topography on a scale exceeding a few nanometers). A smooth membrane has been shown to be inert in the subretinal space, making it suitable for implantation. In addition, as shown herein, culturing RPE cells on a smooth PCL membrane led to well-differentiated cells, as compared with RPE cells cultured on a control transwell chamber or on a nanowire (not smooth) PCL membrane. On a smooth membrane, RPE cells form a characteristic honeycomb pattern with uninterrupted membranous localization of the tight junction marker ZO-1. On a nanowire PCL membrane, the RPE cells were incompletely differentiated, showed signs of apoptosis, including chromatin condensation, and showed discontinuous ZO-1 staining at the junctions and in the cytoplasm (FIGS. 2A-2B). Accordingly, in some implementations, the first (smooth) surface of PCL membrane is free of texture and/or free of structures, such as nanostructures or microstructures, nanowires, topographical or chemical patterns, grooves, microstructures, or scaffolds that form hollow cell culture chambers. In some implementations, the first (smooth) surface is treated with one or more compounds which aid in adhering the cell to the smooth surface. By way of example but not by way of limitation, in some implementations, plasma treatment is used. Additionally or alternatively, in some implementations, oligopeptides are used. In some implementations, one or more oligopeptides that are non-immunogenic and/or that are known to promote cell adhesion when bound to a polymer surface are employed. In some implementations, the peptide comprises arginine-glycine-aspartic acid (RGD) and/or tyrosine-isoleucine-glycine-serine-arginine (YSGSR). In certain implementations, a monolayer of RPE cells grows on the smooth surface of the membrane. The monolayer of cells may be evenly distributed, form tight junctions, and/or have microvilli evenly distributed on the cell surface.

In some implementations, a first surface of the membrane is smooth and a second (e.g., obverse) surface of the membrane comprises an anchoring structure. The anchoring structure may be a textured surface, such as a nanostructure or a microstructure. Exemplary implementations of nanostructures include nanowires (e.g., comprising the same material as the membrane, such as PCL), in which the nanowire structures lie roughly perpendicular to the surface of the membrane, like villi. In some implementations, the anchoring structures promote adhesion to ocular tissue, for example, the Bruch's membrane or other underlying tissue in a patient. The morphology of the second surface may be tuned for optimal adhesion properties to a specific tissue. In some implementations, the anchoring structure reduces slippage or other movement of the membrane in the patient's eye. In certain implementations, the anchoring structure may also comprise a surface-modifed layer that enhances biocompatibility. For example, a nanowire structure may prevent a patient's inflammatory cells from reacting with the membrane after the membrane has been implanted.

The anchoring structure may be used alone, or may be used in combination with adhesive molecules. Adhesive molecules include biocompatible glues, such as acrylate glue and other adhesive substances that bind to ocular tissues, or biologically-derived adhesive molecules, such as proteins from the extracellular matrix. In some implementations, the anchoring structure may be oxygen-plasma-treated or chemically functionalized, e.g., with laminin, fibronectin, vitronectin, RGD (arginine-glycine-aspartate) or other protein sequences.

In further implementations, the porous membrane comprising a first smooth surface and a second surface with an anchoring structure may additionally release therapeutic agents into a biologic environment. In some implementations, the therapeutic agent may be an immunosuppressive agent that reduces an immune response, for example, by downregulating the response of inflammatory cells or by inducing apoptosis of inflammatory cells. In certain implementations, the therapeutic agent is a neuroprotective agent that promotes survival and/or reduces degeneration of retinal neurons. A therapeutic agent may also inhibit angiogenesis, for example, to counteract the choroidal new vessel (CNV) growth under the fovea in AMD patients. An exemplary therapeutic agent may reduce activity of vascular endothelial growth factor (VEGF), for example, by binding to the receptor site of active forms of VEGF and preventing interaction of VEGF with its receptors. Yet another exemplary therapeutic agent is Pigment Epithelial Derived Factor (PEDF), which may be used, for example, to promote development or function of neurons such as photoreceptor cells. Other agents include, without limitation, thrombospondin 1 (a potent anti-inflammatory and anti-angiogenic factor), anti-inflammatory cytokines such as IL-1ra, IL-6, Fas ligand or TGF-beta and neurotrophic/neuroprotective growth factors including, but not limited to glial cell line—derived growth factor, brain-derived neurotrophic factor, nerve growth factor, neurotrophin-3, -4/5, -6, and vitamin E. Such agents may be provided singly or in combination.

In some implementations, the therapeutic agents may be bound to the membrane, or may be released in a sustained manner over time. A number of mechanisms can be utilized for sustained delivery of molecules, including: bulk elution from the material, defined depots of drug, micro/nanospheres, hybrid polymer systems, nano controlled delivery.

### 2. Retinal Pigment Epithelium Cells

One aspect of the invention relates to a composition for improving or restoring sight in patients suffering from AMD or other diseases associated with retinal degeneration, for example, wherein the composition replaces RPE cells that are lost and/or dysfunctional. The composition comprises a membrane as described above and an adherent layer of RPE cells. The membrane may be used alone, or may be used in combination with additional biological substrates such as matrix proteins, collagen, gelatin, basement membrane from the lens, and amniotic membrane.

In some implementations, the RPE cells are mammalian cells, such as human cells or mouse cells. In other implementations, the RPE cells are not mammalian cells, but may be modified to be compatible with mammalian hosts. One exemplary source of RPE cells are immortalized human cell lines, such as ARPE-19 cells. Other exemplary sources for RPE cells include autologous, allogenic, fetal donor, adult donor, and stem-cell derived RPE cells.

In some implementations, stem cell-derived RPE cells may be used in the present composition. Previous studies in animal models indicate that stem-cell derived RPE cells preserves vision if the cells are surgically inserting into the retinas before photoreceptor degeneration occurs. By using induced stem cells that can be derived from patients, the immune rejection that occurs with the use of donor transplant tissue may be avoided. Accordingly, exemplary stem cells may be selected from embryonic, placental, umbilical, mesenchymal, progenitor, or induced pluripotent stem cells. Stem cells may be used for transplantation, for example, by adhering the stem cells to a porous PCL membrane and implanting the membrane into one or both eyes of a patient. Alternatively, stem cells may be induced to differentiate into RPE cells, either before or after attaching the cells to a membrane.

Regardless of the source, the RPE cells adhering to the membrane may express genes characteristic of differentiated RPE. Genes may be selected from at least one of RPE65 (a component of the visual product recycling process in PRE cells), Otx2, Tfeb, Na+K+ pump, CRALBP, PEDF, and VEGF. The RPE cells may also grow in a honeycomb pattern, and/or exhibit localization of the tight junction marker ZO-1 in the membrane of the cells, rather than in the cytoplasm. Markers may be detected at the RNA and/or protein level by methods well known in the art. Finally, RPE cells may be phagocytic.

In some implementations, the cells adhering to the membrane exhibit enhanced growth and differentiation characteristics as compared to cells adhering to or grown on a standard transwell surface, e.g., polyester (“PET”), polycarbonate (“PC”), or collagen-coated polytetrafluoroethylene (“PTFE”). By way of example, but not by way of limitation, in some implementations, cells (e.g., RPE cells) cultured on, or in contact with a membrane of the present technology exhibit one or more of the following characteristics, compared to comparable control cells cultured in a standard transwell, under the same conditions: enhanced RPE65 expression, enhanced CRALBP expression, DEDF expression, VEGF secretion, enhanced tight junction formation, enhanced ZO-1 cell surface localization, increased transepithelial resistance (“TER”), and/or increased phagocytosis.

For example, in some implementations, cells (e.g., RPE cells) in contact with a membrane of the present technology express about 2-fold, 3-fold, 4-fold, 5-fold or about 6-fold higher levels of RPE65 as compared to comparable controls cells cultured in standard transwells. In some implementations, cells (e.g., RPE cells) in contact with a membrane of the present technology exhibit about 100%, 150%, 200%, 250%, 300%, 350%, 400%, 450%, 500%, 550%, 600% or about 650% higher PRE65 expression as compared to comparable control cells cultured in PET transwells. In some implementations, cells (e.g., RPE cells) in contact with a membrane of the present technology express about 50%, 60%, 70%, 80%, 90%, 100%, 110%, 120%, 130%, 140% 150%, 160%, 170% 180%, 190% or about 200% more CRALBP as compared to comparable controls cultured in standard transwells.

### 3. Methods for Treating a Retinal Disorder

A certain aspect of the invention relates to a method for treating symptoms of a retinal disorder in a patient, for example, by implanting in one or both eyes the composition of RPE cells and porous membrane described herein. Retinal disorders may include age-related macular degeneration (AMD) or retinitis pigmentosa. Symptoms of AMD include, for example, choroidal new vessel growth (CNV), atrophy of the fovea, atrophy of the subfoveal retinal pigment epithelium, atrophy of the choroid, and/or loss of central vision. In the early stages of AMD, it may sufficient to replace only RPE cells, for example, by implanting a composition comprising RPE cells and a porous PCL membrane into the eye of a patient. The implanted RPE cells may mediate prevention of further loss of RPE cells and/or degeneration of the Bruch's membrane. At advanced stages of AMD, patients may experience loss of both RPE cells and photoreceptor cells. Thus, in some implementations, the methods described herein may further comprise implanting compositions comprising RPE cells, photoreceptor cells, and a porous PCL membrane.

In some implementations, symptoms of retinal disorders may be treated by a method comprising: (a) obtaining one or more stem cells from the patient; (b) inducing the one or more stem cells to differentiate into retinal pigment epithelium (RPE) cells; (c) contacting the RPE cells with a porous poly(ε-caprolactone) (PCL) membrane, whereby the RPE cells adhere to the porous PCL membrane; and (d) implanting the porous PCL membrane and RPE cells from step (c) into one or both eyes of a patient suffering from a retinal disorder. Exemplary stem cells may be selected from embryonic, placental, umbilical, mesenchymal, progenitor, or induced pluripotent stem cells. Stem cells may be induced to differentiate into RPE cells by methods known in the art, for example, culturing the stem cells in a differentiation medium.

As indicated, the pores of the membrane may be less than 1.0 μm in diameter, for example, within a range of 0.1-1.0 μm, or within a range of 300-500 nm. A first surface of the porous membrane may be used as a surface to culture cells for transplantation. In order to support tight junction formation during growth of a monolayer of RPE cells, the first surface may be smooth, unpatterned, and free of texture or structure. Meanwhile, a second surface may comprise an anchoring structure, which adheres to an ocular structure such as Bruch's membrane. In addition, the porous membrane may additionally release one or more therapeutic agents before, during, or after implantation. One possible outcome after implantation of the composition comprising RPE cells and a porous membrane is restoration of central vision in a patient in need thereof.

The porous membrane may be biodegradable. In some implementations, the membrane degrades slowly, over a span of a few years, during which time RPE cells growing on the membrane may generate their own matrix. The matrix produced by RPE cells may replace a distorted or defective Bruch's membrane. In some implementations, for example, if neovascularization has occurred in the eye of a patient suffering from AMD, the damaged Bruch's membrane may be removed, and the patient may instead rely on the implanted composition as a substitute. The implanted composition may comprise RPE cells attached to an intact porous PCL membrane, or, alternatively, the implanted composition may comprise RPE cells that have produced their own matrix, whether or not the PCL membrane has fully or partially biodegraded.

The size of the composition to be implanted may be generally determined by comparing the clinical assessment of the size of the region of retinal pathology present in a particular patient, with the constraints imposed by surgical feasibility of delivering an implant of a particular size. For example, in degenerations involving the central retina (e.g., age-related macular degeneration), a circular implant of from about 1.0-2.5 mm diameter (e.g., of about 1.5 mm diameter) that approximates the anatomic fovea will frequently be appropriate. In some cases, larger implants may be appropriate, maximally corresponding to the area of posterior retina lying between the temporal vascular arcades (histologic macula, clinical posterior pole) which is an ovoid area of approximately 6.0 mm (vertical)×7.5 mm (horizontal) centered on the fovea. In some instances, it may likewise be appropriate to fashion a polymer scaffold of smaller dimension, as small as about 0.5 mm, to be placed in an area of circumscribed pathology. In addition, it may be of interest to custom fashion implants of irregular shape to suit the patient, for instance to cover areas of pathology while avoiding areas of residual high vision.

In some implementations, the composition to be implanted is coated with a hydrogel prior to implantation. Non-limiting examples of natural polymer hydrogel used for transplant coating include agarose, collagen I, gelatin, and HAMC (hyaluronan and methylcellulose blend). In some implementations, hydrogel composition are selected to allow for complete gelation in less than about 60 minutes, less than about 90 minutes, less than about 2 hours or less than about 3 hours. As is known by those of skill in the art, gelation protocols vary with the polymer composition, and are standard in the art. In some implementations, the hydrogel protect the cells/PCL composition prior to, during, and/or following the implant procedure. In some implementations, the hydrogel degrades shortly after implantation. In some implementations, the hydrogel degrades in about 15 minutes after implant, in about 30 minutes after implant, about 60 minutes after implant, about 1, 2, 3, 4, 5, 6, 10, 12, 20, 24 or about 48 hours after implant. In some implementations, the hydrogel degrades in about 3 days, 5 days 7 days or about 2 weeks after the composition is implanted. In some implementations, hydrogel degradation is from about 1 to 3 days. In some implementations, degradation time varies with the duration of the subretinal bleb (detachment) created during transplantation. Thus, in some implementations, hydrogel degradation is matched to the estimated time needed for bleb resorption and retinal reattachment.

4. Manufacture of a Composition Comprising RPE cells and a Porous PCL Membrane

A further aspect of the present invention is a method for manufacturing a composition comprising RPE cells and a porous membrane. In some implementations, the method comprises contacting RPE cells with a membrane, whereby the RPE cells adhere to the membrane. The porous membrane may be manufactured using clean room microfabrication methods and/or other methods including replica molding, conventional machining, injection molding, sacrificial molding, material printing, laser machining, or solid freeform fabrication.

The membrane upon which RPE cells are attached contains pores, for example, submicron pores with a diameter of 0.1-1.0 μm. In some implementations, the pores are evenly distributed over the surface of the membrane. Pores may be fabricated in a membrane using a templating process. High aspect ratio conical structures are fabricated on silicon using as series of photolithography and etching steps. Using a spin-assisted solvent casting method, the inverse features can be transferred into a polymer thin film. In other implementations, a block copolymer mask may be fabricated and used to protect areas of thin-film during the pore etching process. Both of these methods produce ordered pores within the needed size range, for example, pores of less than 1.0 μm in diameter, within a range of 0.1-1.0 μm, or within a range of 300-500 nm.

In other implementations, the porous membrane may be fabricated, for example, by coating a suitable material onto a wafer, curing it, if applicable, and peeling it off. Pores may then be mechanically punched into the membrane. Alternatively, the membrane may be lithographically patterned, like the other polymer layers. Another fabrication approach involves electrospinning of a membrane with desired porosity, thickness, and other desired properties. Alternatively, a commercially available membrane (e.g., a track-etched polycarbonate membrane from Sterlitech, Kent, Wash.) or a membrane fabricated in situ may be used.

The RPE cells may adhere to a first surface of the porous membrane which is smooth (i.e., has a no surface topography on a scale exceeding a few nanometers). A second surface of the porous membrane may comprise an anchoring structure. The anchoring structure may be a textured surface, such as a nanostructure or a microstructure. Exemplary implementations of nanostructures include nanowires comprising PCL, in which the nanowire structures lie roughly perpendicular to the surface of the membrane, like villi.

Accordingly, in various implementations, the anchoring structure is micro- or nano-patterned. The patterning may be uniformly applied over the entire surface area, or selectively to certain portions of the surface area only. Surface patterning may be achieved during the fabrication of the membrane, using techniques such as, for example, multi-layer photolithographic patterning, a combination of photolithography and etching, transfer molding, 3D printing, or flow lithography. Alternatively or additionally, surface patterning may be applied after completion of the membrane manufacturing process. For example, surface portions (e.g., the inner surfaces of pores in a particular region) may be chemically treated to modify their adhesive properties, conjugate therapeutic components to the surface, etc.

The membrane may release a therapeutic agent into a biologic environment, for example, the eye. In some implementations, the therapeutic agent may be an immunosuppressive agent that reduces an immune response, for example, by downregulating the response of inflammatory cells or by inducing apoptosis of inflammatory cells. In certain implementations, the therapeutic agent is a neuroprotective agent that promotes survival and/or reduces degeneration of retinal neurons. A therapeutic agent may also inhibit angiogenesis, for example, to counteract the choroidal new vessel (CNV) growth under the fovea in AMD patients. An exemplary therapeutic agent may reduce activity of vascular endothelial growth factor (VEGF), for example, by binding to the receptor site of active forms of VEGF and preventing interaction of VEGF with its receptors. Yet another exemplary therapeutic agent is Pigment Epithelial Derived Factor (PEDF), which may be used, for example, to promote development or function of neurons such as photoreceptor cells. Other exemplary, non-limiting agents include thrombospondin 1 (a potent anti-inflammatory and anti-angiogenic factor), anti-inflammatory cytokines such as IL-1ra, IL-6, Fas ligand or TGF-beta and neurotrophic/neuroprotective growth factors including, but not limited to glial cell line-derived growth factor, brain-derived neurotrophic factor, nerve growth factor, neurotrophin-3, -4/5, -6, and vitamin E. Such agents may be provided singly or in combination. A number of methods may be used to direct the controlled release of therapeutics from the membrane, for example, a pump mechanism, controlled nano delivery, or a reservoir system may be used.

RPE cells may be seeded into the membrane utilizing conventional seeding techniques, backfilling, or encapsulation within a secondary gel matrix. Conventional seeding includes delivering the cells to the device by injection or flow, and allowing the cells to statically adhere. Backfilling includes the use of vacuum to disperse cells evenly. Encapsulation involves delivering the cells with a gel in order to distribute cells evenly in a three-dimensional matrix.

In some implementations, stem cells may be induced to differentiate into RPE cells before the RPE cells are seeded, attached, and/or cultured on the membrane. In other implementations, stem cells may be seeded, attached, and/or cultured on the membrane and induced to differentiate into RPE cells on the membrane. Exemplary stem cells may be selected from embryonic, placental, umbilical, mesenchymal, progenitor, or induced pluripotent stem cells.

In some implementations, the cells and the membrane scaffold are coated with a hydrogel.

In some implementations, RPE cells and/or tissues cultured on a porous membrane may be used as model systems to study RPE biology, as well as mechanisms of retinal damage and disease, toxic retinopathies, and/or ocular neovascularization. Substances that promote neuroprotection or neuroregeneration, as well as substances that reduce angiogenesis may also be tested on the compositions described herein.

### EXAMPLES

Having generally described the invention, Applicants refer to the following illustrative examples to help to understand the generally described invention. These specific examples are included merely to illustrate certain aspects and implementations of the present invention, and they are not intended to limit the invention in any respect. Certain general principles described in the examples, however, may be generally applicable to other aspects or implementations of the invention. The invention contemplates that any one or more of the aspects, implementations and other features described above and below can be combined.

### Example 1

### Growth and Differentiation of Human RPE on PCL Membranes

Studies were conducted using ARPE-19 cells, a line of spontaneously immortalized human RPE that have been documented to be a reliable model of RPE differentiation (Dunn, 1996 #1167; Dunn, 1998 #1168). First, their adhesion to smooth and nanowire PCL polymer was examined. ARPE-19 cells were plated at confluence (1.7×10⁵ cells/cm²) on laminin-coated polyester membranes in transwell chambers (Costar) (control), PCL smooth, or PCL nanowire polymers pre-coated with laminin and inserted into transwells.

PCL pellets of 70,000-90,000 Da (Sigma-Aldrich, St. Louis, Mo.) were dissolved 2:15 (w/v) in dichloromethane (Sigma-Aldrich, St. Louis, Mo.) with vigorous stirring for three hours at room temperature. Thin films were fabricated using a spinning process to achieve near-uniform thickness. Briefly, 10 ml of the PCL solution was deposited onto a 100 mm-diameter polished silicon wafer and quickly spun at 1,500 RPM for 30 seconds to produce a solid PCL film adherent to the wafer. The PCL and wafer were then heated at 60° C. to allow the polymer to melt and reflow into locally uneven areas. The PCL solution was cast onto a nanoporous anodized aluminum oxide template using a spin coater (Specialty Coating Systems, Indianapolis, Ind., USA). The solvent was allowed to evaporate at room temperature. Polymer melts were formed at 130° C. while in contact with the nanoporous template. Nanowire length was tuned as a function of melt time. A melt time of 5 minutes formed nanowires 2.5 μm in length, while a melt time of 60 min formed nanowires 27.5 μm in length. The thin-film scaffold of vertically aligned nanowires was released by etching the template in a dilute sodium hydroxide solution and allowed to air dry at room temperature.

ARPE-19 cells (ATCC) were grown in DMEM/F-12 (Lonza) supplemented with 10% FBS (Atlanta), Glutamax (Gibco), and penicillin-streptomycin (Lonza), in plastic flasks (BD), and incubated at 37° C., 5% CO₂. ARPE-19 cells were plated at high density (1.7×10⁵ cells) on 0.4 μm-pore 12-well Costar polyester (PET) transwells (Thermo Fisher Scientific, Cambridge, Mass.) or on PCL films mounted on an empty transwell support and maintained in DMEM/F12 supplemented with 1% FBS, Glutamax and penicillin-streptomycin. Media were changed twice a week for up to six weeks.

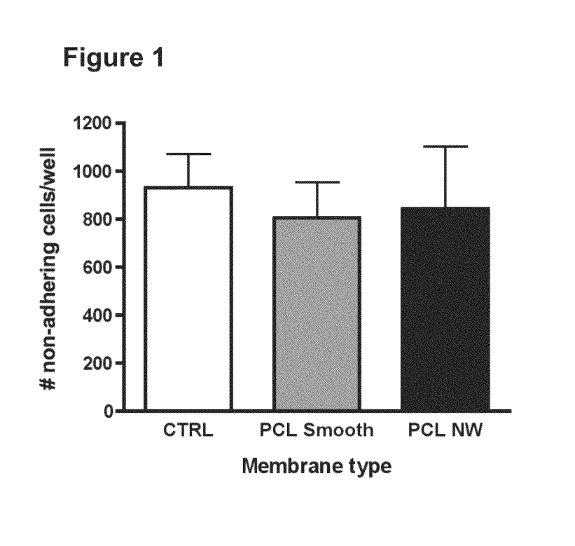

The cells were left on the inserts overnight, and the number of non-adherent cells was counted by collecting the conditioned media from each sample. The number of attached cells was identical for the control, PCL smooth and PCL nanowire transwells (FIG. 1).

The ability of ARPE-19 cells to differentiate on smooth and nanowire membranes was assessed. APRE-19 cells were plated at confluence, allowed to grow for one week, then fixed and stained for the tight junction marker ZO-1 (red), F-actin (green) and DAPI (blue). On the smooth PCL, cells appeared already well differentiated and formed a characteristic honeycomb pattern with membranous localization of ZO-1 while on control or on nanowire PCL membrane, RPE differentiation was incomplete (FIG. 2A). RPE grown on the nanowire PCL showed signs of apoptosis including chromatin condensation (arrows).

The ability of ARPE-19 to maintain a differentiated phenotype after 6 weeks of differentiation was characterized by confocal analysis of ZO1 localization (FIG. 2B). On smooth PCL, ZO1 staining was perfectly uninterrupted and absent from the cell cytoplasm. On plastic transwells, ZO1 staining appeared discontinuous at the junctions (arrows) and present in the cell cytoplasm (asterisks).

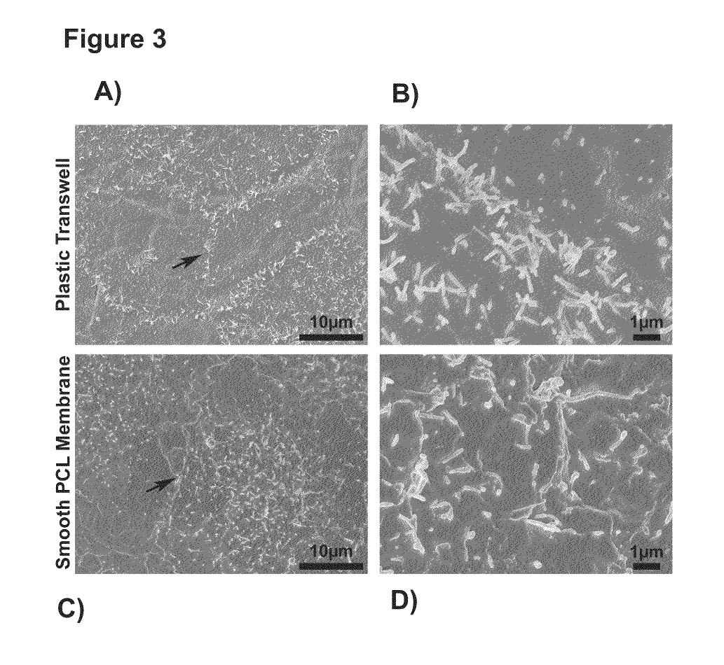

The structural morphology of ARPE-19 cells cultured on control or smooth PCL membranes for six weeks were examined by SEM (FIG. 3). RPE cells grown on the PCL smooth membrane formed a very flat monolayer, as evidenced by the intimate cell association (black arrow). On the plastic transwell, the microvilli appeared preferentially distributed at the cell junctions (white arrows). On the smooth PCL scaffold, microvilli were evenly distributed on the cell surface.

### Example 2

### Effects of Culturing Human RPEs on Smooth PCL Membranes

The expression of genes that characterize differentiated RPE was examined. RNA was collected from ARPE-19 cells cultured on transwell membranes or on smooth PCL for one and four weeks and the expression of genes associated with RPE differentiation and function were analyzed by quantitative PCR.

Total mRNA was purified using RNA-Bee solution (IsoText Diagnostic, Inc., Friendswood, Tex.) under RNAse-free conditions, according to the manufacturer's instructions. One μg of RNA was reverse-transcribed using Superscript III (Invitrogen, Carlsbad, Calif.). Reactions were performed using the SYBR Green Master mix and the LightCycler 480 Real-Time PCR System (Roche Applied Science, Indianapolis, Ind.) according to the manufacturer's instructions. Primer sequences are listed in Table 1.

| Gene | Forward primer | Reverse primer |
|---|---|---|
| TFEB | CGCATCAAGGAGTTGGGAAT | CTCCAGGCGGCGAGAGT |
| (transcription factor | | |
| EB) | | |
| Ezrin | GTTTTCCCCAGTTGTAATAGTG | TCCGTAATTCAATCAGTCCT |
| | CC | GC |
| Otx2 | TAAGCAACCGCCTTACG | GCACTTAGCTCTTCGATT |
| (orthodenticle | | |
| homeobox 2) | | |
| MITF | AGCCATGCAGTCCGAAT | ACTGCTGCTCTTCAGCG |
| (microphthalmia- | | |
| associated | | |
| transcription factor) | | |
| CRALBP | GCTGCTGGAGAATGAGGAAAC | TGAACCGGGCTGGGAAGGA |
| (cellular | T | ATC |
| retinaldehyde- | | |
| binding protein) | | |
| NaK Pump | ACAGCCTTCTTCGTCAGTATCG | CGAATTCCTCCTGGTCTTAC |
| (Na+/K+ ATPase) | T | AGA |
| VEGF | GGGCAGAATCATCACGAAGTG | ATTGGATGGCAGTAGCTGCG |
| (vascular endothelial | | |
| growth factor) | | |
| PEDF | TATCACCTTAACCAGCCTTTCA | GGGTCCAGAATCTTGCAATG |
| (pigment epithelium- | TC | |
| derived factor) | | |
| FGF-2 | GCGACCCACACGTCAAACTA | TCCCTTGATAGACACAACTC |
| (basic fibroblast | | CTC |
| growth factor) | | |
| HPRT | TCAGTCAACGGGGGACATAAA | GGGGCTGTACTGCTTAACCA |
| (Hypoxanthine- | | G |
| guanine | | |
| phosphoribsyltrans- | | |
| ferase) | | |
| GAPDH | CCCATCACCATCTTCCAGGA | CATCGCCCCACTTGATTTTG |
| (glyceraldehyde 3- | | |
| phosphate | | |
| dehydrogenase) | | |

Each sample was subjected to melting curve analysis to confirm amplification specificity. Each sample was run in triplicate, and each experiment included two non-template control wells. Samples were normalized to the housekeeping genes, GAPDH and HRPT1, and expressed as the relative expression using the delta-delta Ct method.

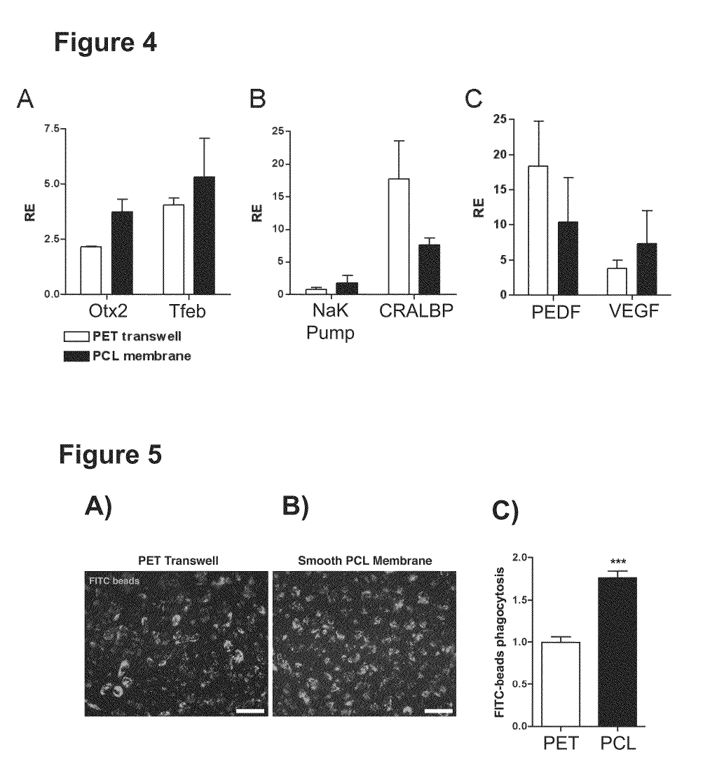

Results are expressed as the ratio compared to undifferentiated, subconfluent ARPE-19 cultured on plastic (n=3). The expression of transcription factors involved in RPE differentiation (Otx2, Tfeb) was significantly upregulated after one and four weeks of culture under both conditions (FIG. 4A). Genes involved in RPE function such as Na⁺K⁺ pump and CRALBP were also analyzed. The Na⁺K⁺ pump was not upregulated in either culture condition whereas CRALBP was strongly induced under both conditions (FIG. 4B). The level of upregulation was reduced in ARPE-19 cells cultured on PCL membrane. Secreted factors, PEDF and VEGF, were similarly upregulated by ARPE-19 cells cultured on transwell and PCL membrane (FIG. 4C).

The function of ARPE-19 cells cultured on smooth PCL membranes was further assessed by examining their phagocytic activity. ARPE-19 cells were differentiated for 4 weeks on PET transwell (FIG. 5, left panel) or smooth PCL membrane (FIG. 5, middle panel). FITC-labeled 0.4 μm latex beads were added on the top of the culture chamber at a concentration of 10⁷ beads/ml and the cells were incubated in the presence of the bead for 16 hr. After multiple PBS washes, cells were fixed and photographed under the fluorescent microscope. ARPE-19 cells cultured on PCL showed a significant increase in phagocytic activity, as evidenced by a more intense and uniform FITC staining A comparison of phagocytic activity is summarized in a bar graph (FIG. 5, right panel).

### Example 3

### Methods for Differentiation of RPE Cells from Induced Pluripotent Murine Stem Cells (IPSCs)

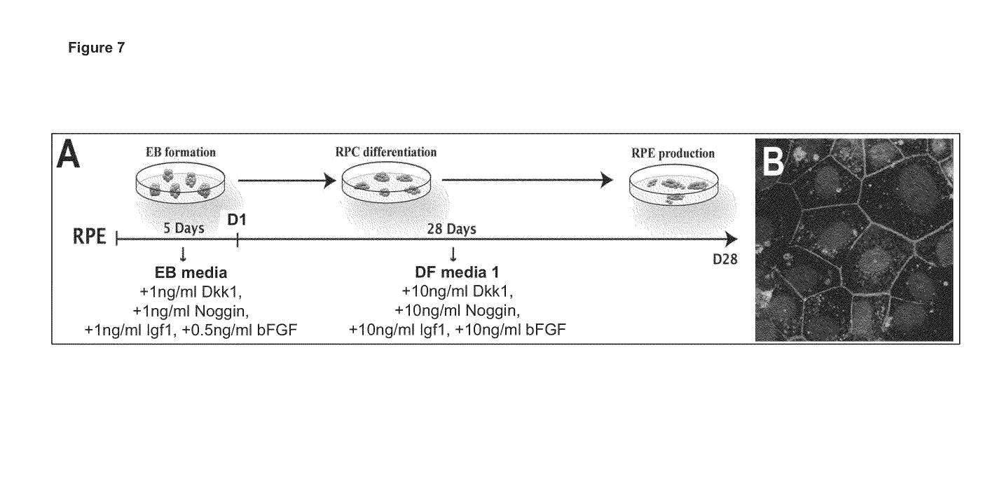

Methods for iPSC generation and RPE cell differentiation, isolation and culture are briefly described herein (FIG. 7). FIG. 7A shows a diagram of the differentiation protocol. FIG. 7B shows ZO-1 positive RPE cells (green: ZO-1, red: dsRed, blue: DAPI).

To maintain pluripotency, adult dsRed-iPSCs were cultured on inactive mouse embryonic fibroblasts in DMEM media (Gibco) containing 15% heat inactivated-FBS (Lifeblood Medical Inc.), 0.0008% β-mercaptoethanol (Sigma-Aldrich), 1% 100×NEAA (Gibco), 1×10⁶ units/L of leukemia inhibitory factor (LIF/ESGRO, Millipore), 1% penicillin/streptomycin (Gibco) and 0.2% fungizone (Gibco). Cells were maintained at 37° C. at 5% CO₂. As shown in FIG. 7, to induce differentiation, iPSCs were removed from the culture substrate via incubation in a 1 mg/ml type I collagenase (Sigma-Aldrich) solution, resuspended in embryoid body media [DMEM F-12 media (Gibco) containing 10% knockout serum replacement (Gibco) 2% B27 supplement (Gibco) 1% N2 supplement (Gibco), L-Glutamine (Gibco), 1% 100×NEAA (Gibco), 1% penicillin/streptomycin (Gibco), 0.2% fungizone (Gibco), 1 ng/ml Noggin (R&D Systems, Minneapolis, Minn.), 1 ng/ml DKK1 (R&D Systems), 1 ng/ml IGF-1 (R&D Systems) and 0.5 ng/ml bFGF (R&D Systems)], and plated at a density of ˜500 cell clumps/well in 6-well ultra low cluster plates (Corning, Lowell, Mass.). Cell clumps are cultured for five days at 37° C. at 5% % CO₂, then embryoid bodies are removed, washed and plated in fresh differentiation media 1 [DMEM F-12 media (Gibco), 2% B27 supplement (Gibco) 1% N2 supplement (Gibco), L-glutamine (Gibco), 1% 100×NEAA (Gibco) 10 ng/ml Noggin (R&D Systems), 10 ng/ml DKK1 (R&D Systems), 10 ng/ml IGF1 (R&D Systems) and 1 ng/ml bFGF (R&D Systems)] in 6-well culture plates coated with poly-D-lysine (BD Bioscience, San Jose, Calif., 10 μg/ml), Collagen (BD Bioscience, 25 μg/ml), laminin (Gibco, 50 μg/ml) and fibronectin (Sigma-Aldrich, 100 μg/ml). For RPE cell differentiation the cultures were maintained in this media for 28 days at 37° C. at 5% CO₂.

### Example 4

### Growth of RPE Cells Derived from Induced Pluripotent Stem Cells

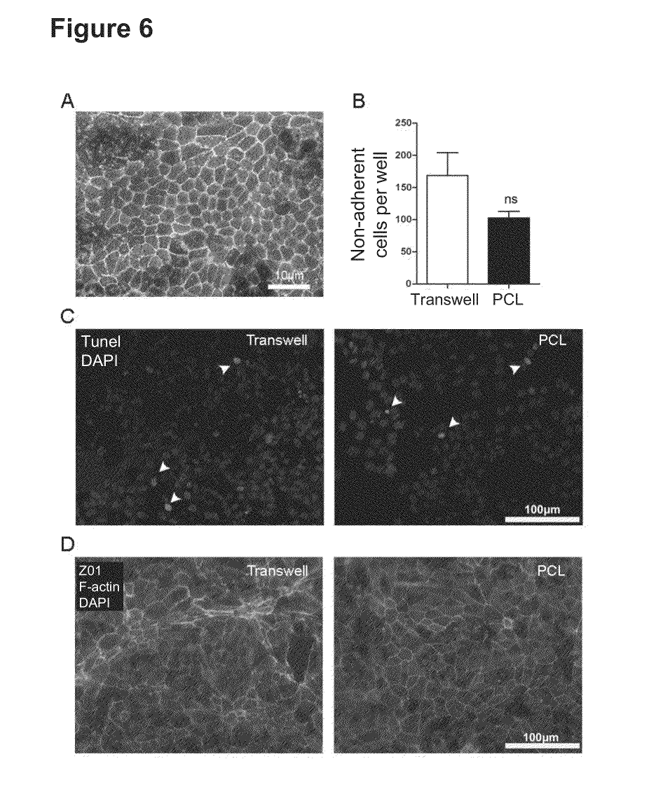

Successful transplantation of RPEs requires RPE cells to be compatible with the intended transplant host. Studies were conducted using RPE derived from the induced pluripotent stem cell (iPSC) (Buchholz, 2009 #6533). Human RPE derived from iPSCs (iPSC-RPE) were cultured and amplified on gelatin-coated dishes at 37° C., 5% CO₂ in Human Fetal RPE medium containing a-MEM, 1×N1 supplement, 1× Non-essential amino acid solution, 250 mg/ml taurine, 13 ng/ml Triiodo thyronin, 20 ng/ml Hydrocortisone, 2 mM L-glutamine, and 15% heat-inactivated foetal bovine serum. At confluence, the cells exhibited a characteristic RPE phenotype with strong, uniform pigmentation and honey-combed morphology (FIG. 6A).

iPSC-RPE adhesion to the transwell and PCL membrane was similar (FIG. 6B). iPSC-RPE were dissociated with 0.05% trypsin EDTA and plated at 3×10⁵/cm² on laminin-coated transwells or PCL films. After one week of culture on transwell or PCL membrane, there was no significant increase in apoptosis detected as Tunel-positive cells (arrowhead) (FIG. 6C). Improved differentiation of the iPSC-RPE on PCL membrane was seen and is demonstrated by the epithelial morphology and ZO-1 localization (FIG. 6D).

### Example 5

### Release of FITC-Albumin from PCL Membranes

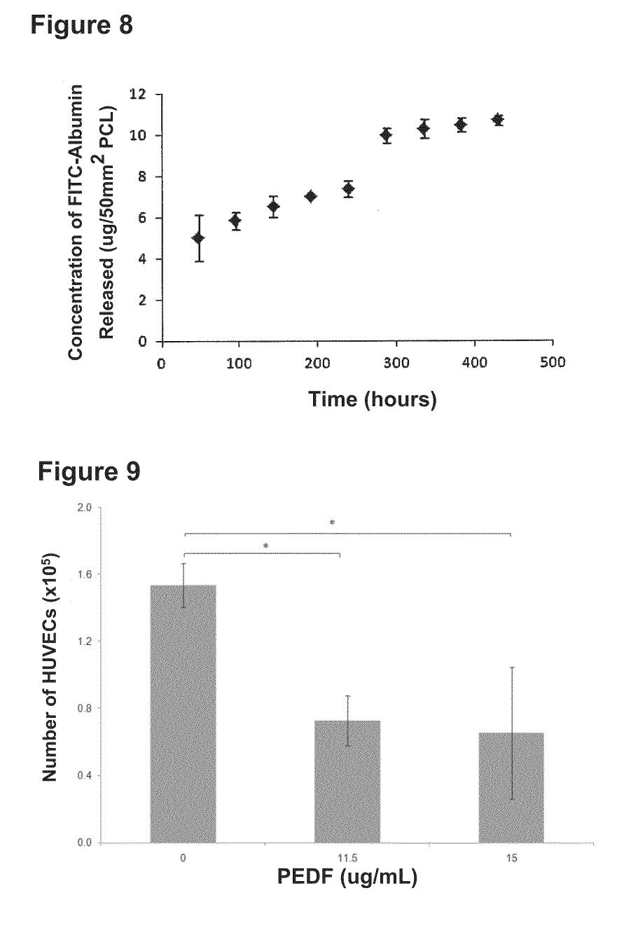

The amount of FITC-albumin released from PCL in PBS over a three-week period was measured. Samples of PBS solution were tested using a spectrophotometer to quantify the amount FITC-albumin released. FIG. 8 shows the release profile. Analysis of release concentrations were performed up to 18 days. Burst release is observed from 0 to 2 days, followed by a period of sustained release from 2 to 10 days. There is another short period of burst release from 10 to 12 days followed by sustained release up to 18 days.

### Example 6

### Effects of Pigment Epithelium Derived Factor on Growth of Human Umbilical Vein Endothelial Cells and Photoreceptors

The effect of Pigment Epithelium Derived Growth Factor (PEDF) on growth of Human Umbilical Vein Endothelial Cells (HUVECs) and on photoreceptors was measured. Human umbilical vein endothelial cells (HUVECs) were maintained in EBM-2 (Lonza, Rockland, Me.) supplemented with SingleQuots (Lonza), 20% fetal bovine serum (FBS), and 1% glutamine-penicillin-streptomycin (GPS) at 37° C. and 5% CO₂. HUVECs were seeded on gelatin coated 12-well plates inserts with a density of approximately 6200 cells/mm² (Costar) and cultured until confluence. PEDF (0, 11.5, and 15 μg/mL) was then added to the medium. 7 days later a LIVE/DEAD cell viability assay (Invitrogen, Carlsbad, Calif.) was used to quantify the number of live HUVECs.

FIG. 9 shows the number of living HUVECs as a function of PEDF concentration (μg/mL). The number of living HUVECs decrease proportionally to the increase of PEDF concentration. * Indicates p<0.05 for the number of HUVEC Cells in media with PEDF compared to media containing no PEDF using student's paired t-test (n=3 samples in each case).

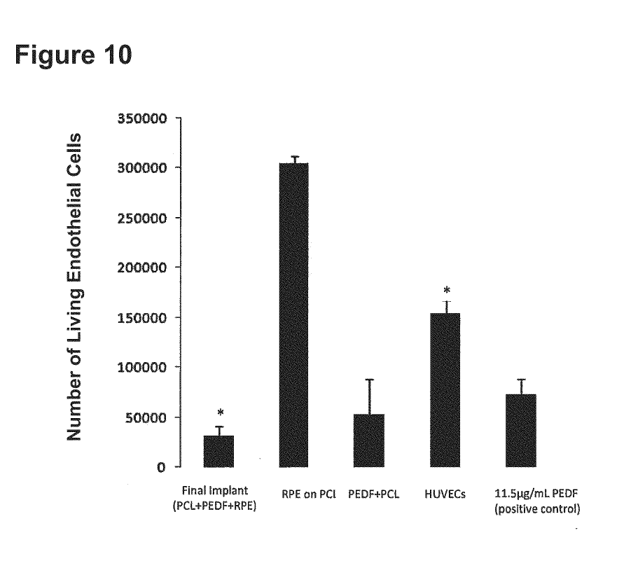

FIG. 10 demonstrates that the PCL-PEDF composite has a significant negative impact on HUVEC proliferation. The presence of PEDF in both the PCL-PEDF and PEDF alone samples also showed decreased numbers of HUVECS following incubation. HUVEC alone and immature RPE on plain PCL allowed for active HUVEC proliferation. * Indicates p<0.05 for the final implant compared to HUVEC Cells in media using a student's paired t-test.

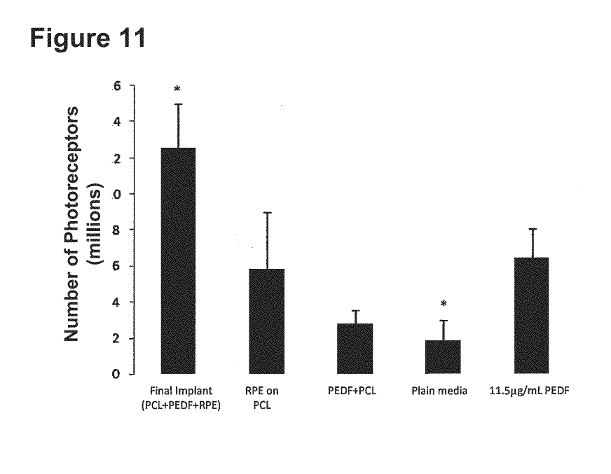

FIG. 11 shows the effects of PEDF on photoreceptors. Analysis of the effect of PEDF on photoreceptor survival shows a significant positive impact. The most significant level of photoreceptor survival was in the PCL-PEDF-RPE condition followed by media containing 11.5 μg/ml PEDF. Photoreceptor survival was similar between RPE-Plain PCL and PCL-PEDF. The lowest photoreceptor survival rate was observed in media in the absence of RPE and PEDF.

* Indicates p<0.1 for the final implant compared to photoreceptors in media using a student's paired t-test.

### Example 7

### Fabricating Porous PCL Membranes

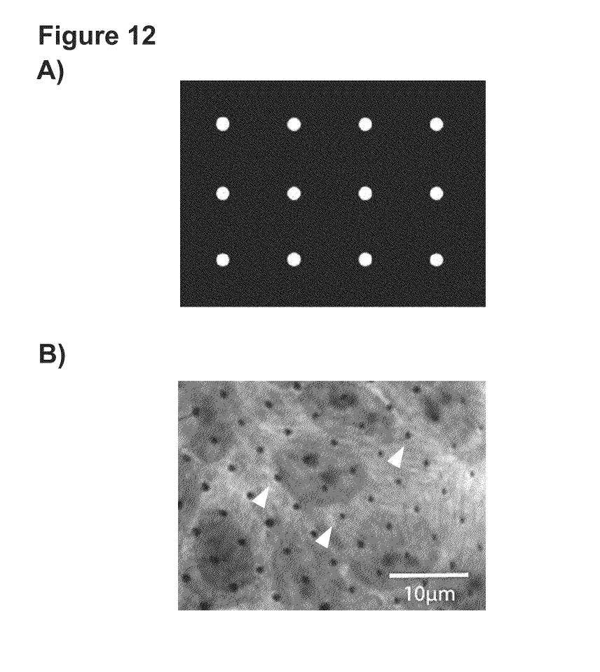

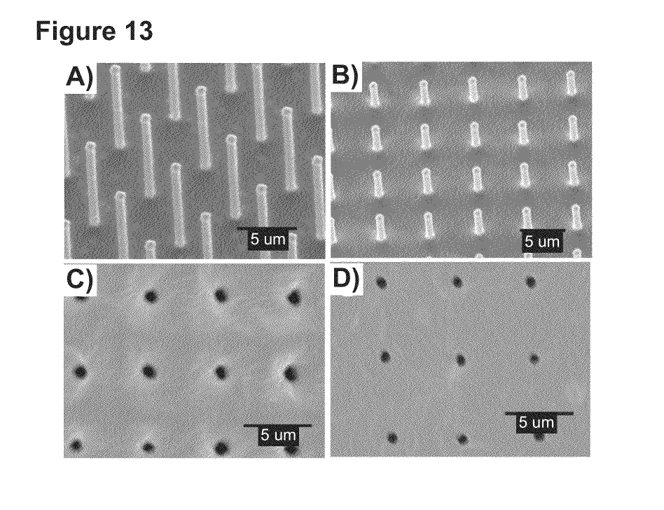

A porous PCL membrane was fabricated using photolithography, reactive ion etching, and spin-assisted solvent casting. First, a computer-aided design program was used to design a photomask pattern of two-dimensional shapes to be projected into a three-dimensional feature using microfabrication techniques. This pattern consisted of repeating 1 μm transparent circles arranged in a square array with 5 μm center-to-center spacing (FIG. 12). Next, photolithography was used to transfer the pattern from the photomask into a negative photoresist coated on the surface of a silicon wafer. Deep reactive ion etching was then employed to selectively etch unprotected silicon and create three-dimensional cylindrical features perpendicular to the wafer surface (FIG. 13A). These cylinders were approximately 14.3 μm in height and 730 nm in diameter with a sidewall angle of 90.1°.

After the mold was fabricated, it was coated with a PCL dissolved in dichloromethane (2:15 weight/volume) and rotated at 1500 RPM to achieve a 9.9 μm-thick film less than the height of the silicon features (FIG. 13B). The film was then carefully peeled from the wafer yielding pores where the features had been. Pores corresponding to the top of the features were 650 nm in diameter (FIG. 13C) while those at the bottom were 810 nm in diameter (FIG. 13D).

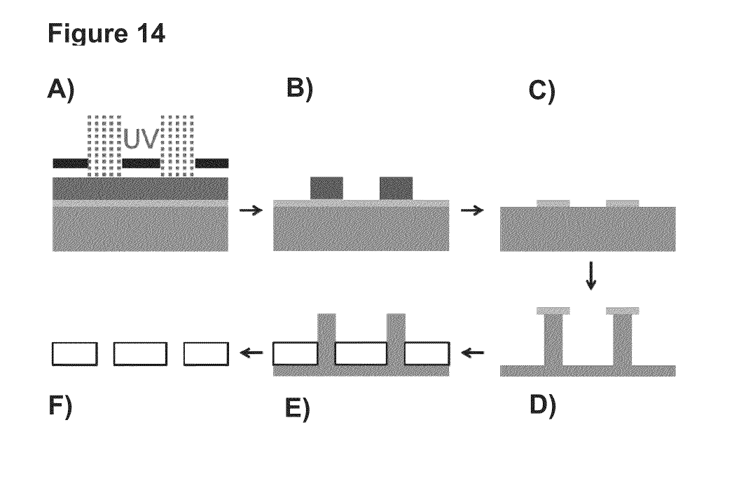

By way of example, but not by way of limitation, a full mold and film fabrication process is summarized in FIG. 14. Briefly, a silicon wafer coated with silicion dioxide and negative photoresist is exposed to deep UV light through a photomask (A). The exposed photoresist remains while the unexposed photoresist is developed away. Reactive ion etching then removes uncovered silicon dioxide, but areas covered by photoresist remain (C). Deep reactive ion etching preferentially removes silicon in a vertical direction (D). Silicon dioxide is removed using a wet etch and the resulting silicon mold is spin-coated with PCL (E). The PCL is removed yielding pores where the silicon cylinders used to be (F).

### Example 8

### Fetal Human RPE on Porous PCL Membranes

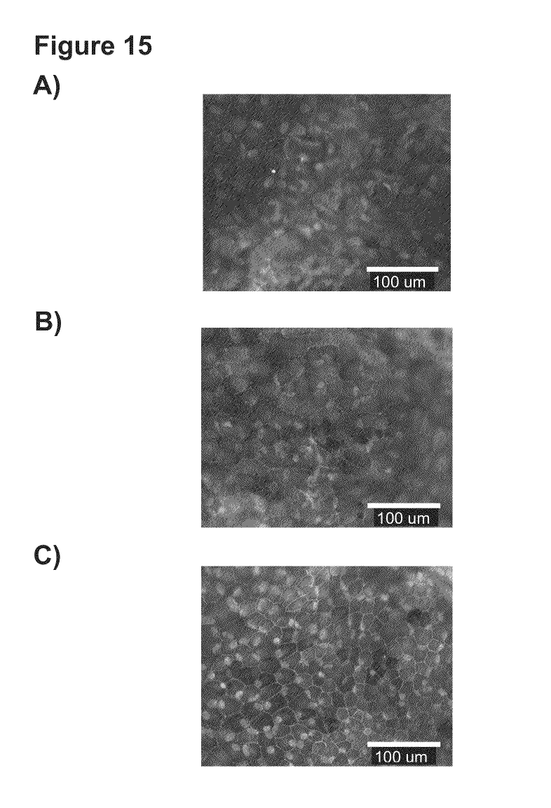

Fetal human RPE cells (Lonza) were cultured on porous PCL membranes for up to 8 weeks and assessed using multiple assays for RPE maturation. Cells were cultured in RtEBM medium (Lonza) containing 2% of FBS. Cells (up to passage 4) were seeded at 3×10⁵ cells/cm² on laminin-coated transwells and maintained in serum free RtEBM medium. Non-porous PCL membranes and polyester transwells were used as control cell substrates for comparison. The greatest degree of pigmentation, a visual marker of RPE maturity, was observed in RPE on porous PCL followed by non-porous PCL and finally polyester transwells beginning at 4 weeks of culture. Cells cultured on the porous PCL scaffold also assumed the characteristic hexagonal shape in greater quantities than on either of the control materials. Immunohistochemical staining for ZO-1, a marker of tight junction, was utilized to characterized cell junction formation. ZO-1 localization at the cell-cell interface was strongly enhanced in cells cultured the porous PCL scaffold (FIG. 15). Briefly, samples were fixed in 4% paraformaldehyde for 10 minutes, blocked with 3% goat serum, 3% donkey serum, 0.1% Tween in PBS for 1 h at room temperature and incubated overnight at 4° C. with rabbit anti-ZO1 (1:100, Invitrogen) followed by Dylight 488-goat anti-rabbit secondary antibody (Jackson, 1:300). Cell nuclei were identified by DAPI (Invitrogen) labeling. Images were taken with a Zeiss Axioscope. This qualitative observation was confirmed using transepithelial resistance (TER), an indicator of monolayer barrier function. Trans epithelial resistance of cell monolayers was monitored with an epithelial voltohmmeter using an Endohm electrode (WPI). TER was significantly increased on porous PCL compared to polyester transwells beginning at 3 days and persisting through the end of the 4 week experiment (FIG. 16). These observations indicate that porous PCL scaffold enhances RPE cells barrier function.

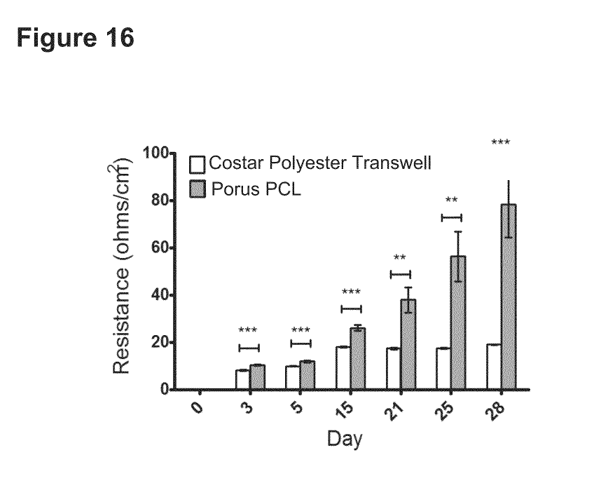

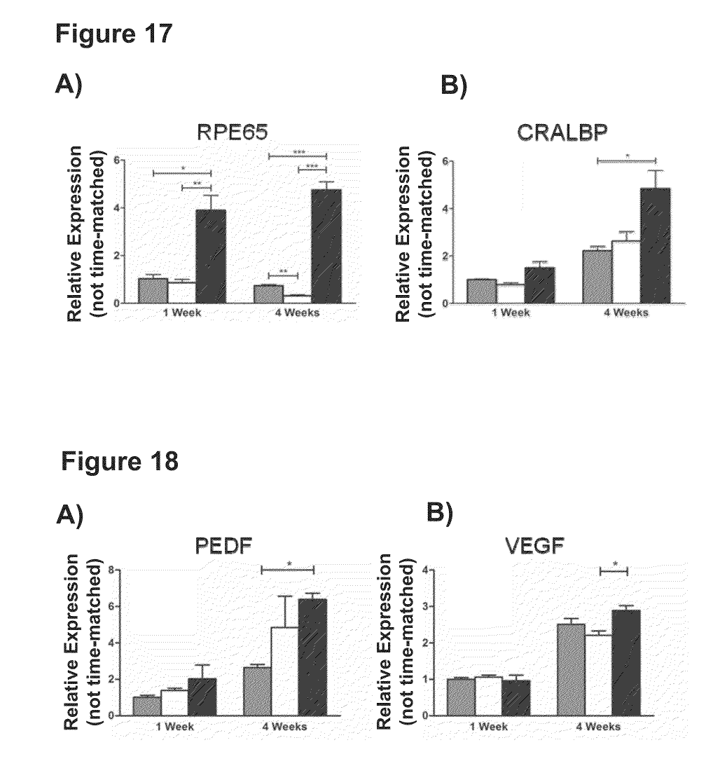

Quantitative PCR identified a significant upregulation of the key visual cycle proteins RPE65 and CRALBP at the mRNA level (FIG. 17). PCR was performed as described previously. Relative expression of RPE65 at 4 weeks in cells on porous PCL exceeded expression on polyester transwells by 550% (p<0.001). Cells on porous PCL similarly expressed more CRALBP, experiencing a 120% increase (p<0.05). Pigment epithelium derived factor (PEDF) and vascular endothelial growth factor (VEGF) are two of the factors secreted by RPE to maintain the adjacent neural and vascular environments respectively. Quantitative PCR revealed a significant increase in PEDF mRNA in RPE cultured on porous PCL compared to cells on the polyester transwell control (p<0.05) at 4 weeks (FIG. 18A) while VEGF mRNA was statistically similar between these groups (FIG. 18B).

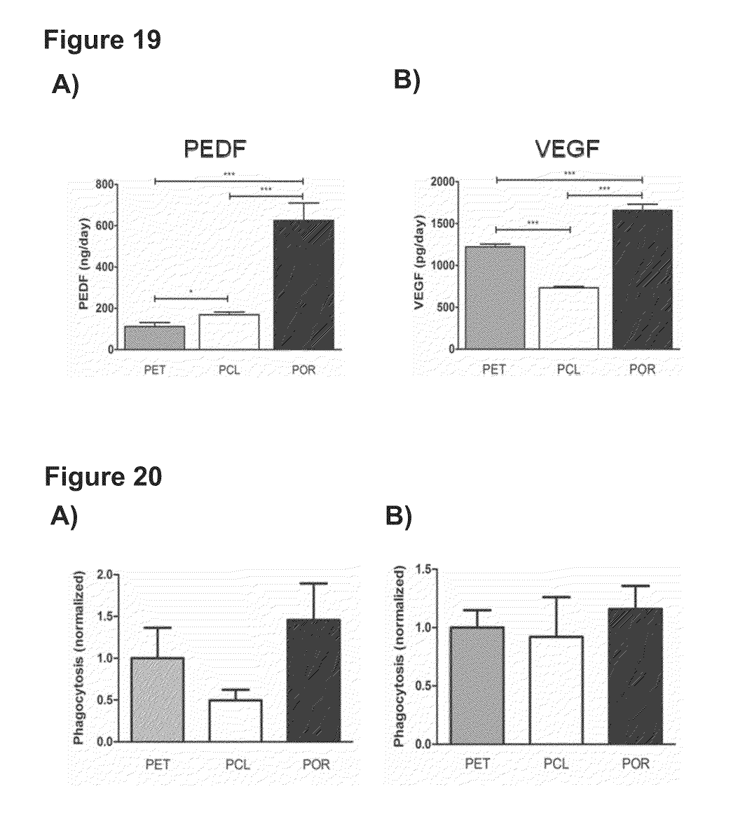

ELISA analysis of conditioned media indicated that RPE cultured on the porous PCL secreted significantly more PEDF (p<0.001) than cells on either of the control materials (FIG. 19A). VEGF and PEDF protein levels in cells conditioned medium was quantified using human ELISA kits following manufacturers instructions (VEGF Sandwich ELISA, R&D Systems and PEDF ELISA, BioProductsMD). Further, VEGF secretion was also seen to significantly increase (p<0.001) compared to either control (FIG. 19B).

Lastly, cells cultured for 4 weeks on porous PCL displayed a trend towards increased outer segment phagocytosis compared to cells on either control substrate, though this was not significant. Phagcytosis activity was determined as follows. Photoreceptor outer segments (POS) were isolated from freshly slaughtered bovine eyes using a continuous sucrose gradient. POS were FITC-labeled in 0.1 M sodium bicarbonate/5% sucrose. 1.6×10⁶ FITC-POS were added to the cells for 18 hours. External fluorescence was quenched with trypan blue for 10 min. Cells were then washed in PBS and processed for immunocytochemistry using anti-rhodospin antibody. Green and red fluorescence intensity corresponding to the phagocytized POS was quantified on 6 fields, representing a total area of 3.54 mm² per transwells, by pixel densitometry using ImageJ software and expressed as compared to control.

Taken together, these results indicate that RPE appear to exhibit superior behavior on porous PCL compared to polyester transwells and non-porous PCL.

### Example 9

### Adhesion-Promoting Oligopeptide Conjugation to PCL

Because synthetic cell culture surfaces lack cell binding domains, in some implementations, the surface is treated prior to cell seeding to promote anchorage-based cell adhesion. Though laminin adsorption is frequently used for this purpose, it is animal-produced and therefore potentially immunogenic. Therefore, the use of only the adhesion promoting peptide sequences from laminin and other extracellular matrix molecules may provide non-immunogenic, therapeutically-superior alternatives. In this experiment oligopeptides containing either the sequence Arginine-Glycine-Aspartic acid (RGD) or Tyrosine-Isoleucine-Glycine-Serine-Arginine (YIGSR) were covalently attached to polyester (a copolymer including PCL) to promote adhesion. These test groups were compared to a laminin-coated and plasma-treated surface.

The oligopeptides RGD (EMD Chemicals) and YIGSR (BACHEM) were conjugated to the surface using the protocol published in 2010 by Causa, et al. in Langmuir, 26(12). Briefly, polymer surface chains of PCL exposed to a diamine solution undergo aminolysis resulting in an exposed primary amine group attached to the polymer backbone. This exposed group was then conjugated to a gluteraldehyde and covalently linked to a peptide motif containing an adhesion domain.

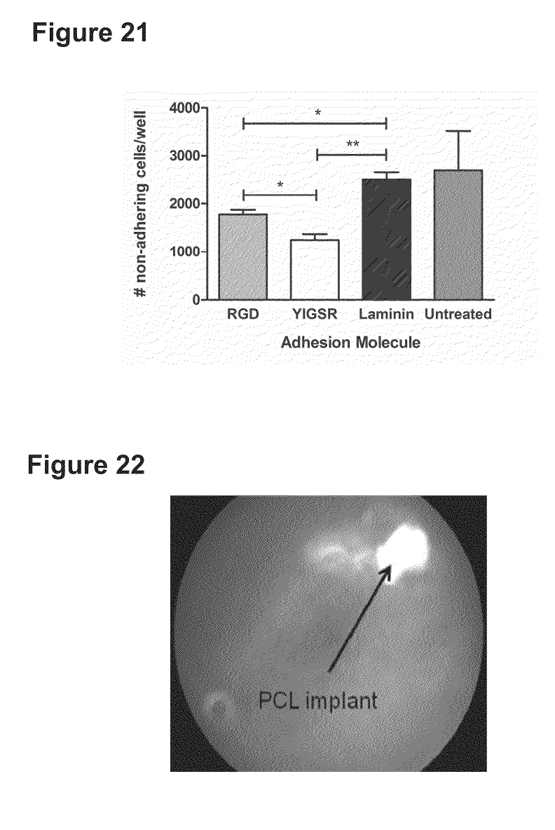

Adhesion assays investigating the number of non-adherent fetal human RPE 24 hours after seeding indicate that conjugated YIGSR promotes adhesion better than both RGD and laminin (p<0.05 and p<0.01 respectively) and that RGD promotes adhesion better than laminin (p<0.05). Cells were seeded at 3×10⁵ cells/cm² on treated or untreated porous PCL film in RtEBM medium plus 2% of FBS. The number of floating (non adherent) cells in medium was quantified in 24 hours after plating. These results are summarized in FIG. 21.

### Example 10

### PCL Film Implantation into a Mouse Model

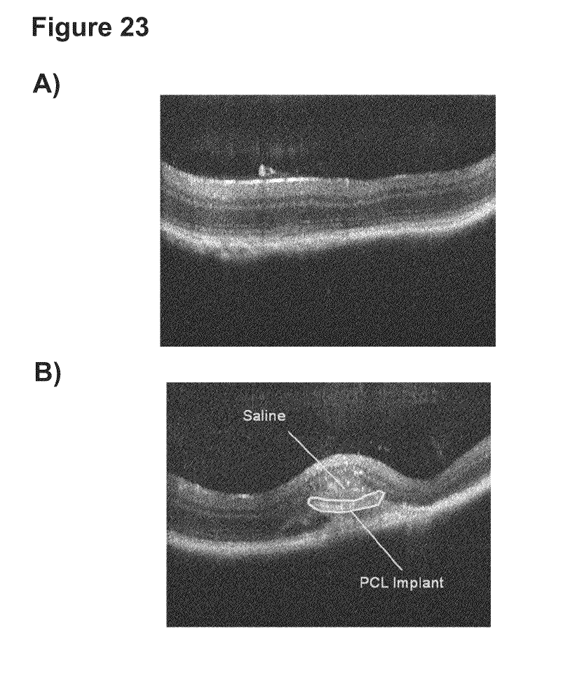

To establish the efficacy of an RPE-porous PCL construct it should first be tested in an animal model for biocompatibility and functionality. In previous studies, PCL has been shown to be well-tolerated in the sub-retina, so no biocompatibility issues are expected. In this experiment a 1.5 mm×1.5 mm piece of naked porous PCL was implanted into the sub-retinal space of C57B1/6J mice in a cavity created by a small volume saline injection. Though the mouse eye is particularly difficult to perform surgery on, initial experiments have shown that it is possible to implant the thin film into the eye (FIG. 22) and access the correct layer of the retina (FIG. 23). Based on fundus analysis, porous PCL transplantation did not elicit ocular inflammation and appeared well tolerated by the retina.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific implementations of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A composition comprising retinal pigment epithelium (RPE) cells adhering to a poly(ε-caprolactone) (PCL) membrane, wherein the PCL membrane comprises a plurality of pores distributed over the PCL membrane, each pore having a diameter of less than 1 micron.

2-3. (canceled)

4. The composition of claim 1, wherein the pores are substantially evenly distributed over the surface of the PCL membrane.

5. (canceled)

6. The composition of claim 1, wherein the PCL membrane is flexible.

7. The composition of claim 1, wherein the PCL membrane comprises a first surface and second surface, and wherein the first surface on the PCL membrane is smooth.

8. The composition of claim 7, wherein a second surface of the PCL membrane comprises an anchoring structure.

9. The composition of claim 8, wherein the anchoring structure is a nanostructure or microstructure that adheres to an ocular tissue.

10. The composition of claim 8, wherein the anchoring structure comprises nanowires.

11. The composition of claim 8, wherein the anchoring structure comprises a surface-modifying layer that enhances biocompatibility.

12. The composition of claim 11, wherein the surface modifying layer comprises ECM molecules.

13. (canceled)

14. The composition of claim 9, wherein the ocular tissue is Bruch's membrane.

15-18. (canceled)

19. The composition of claim 1, wherein the PCL membrane releases a therapeutic agent into a biologic environment.

20. The composition of claim 19, wherein the therapeutic agent induces apoptosis of inflammatory cells, suppresses an immune response, reduces degeneration of retinal neurons, and/or inhibits angiogenesis.

21. The composition of claim 20, wherein the therapeutic agent reduces activity of VEGF.

22. The composition of claim 1, wherein the RPE cells are mammalian cells.

23. (canceled)

24. The composition of claim 22, wherein the RPE cells are human cells selected from the group consisting of fetal cells, adult cells, immortalized cells and stem cells.

25-32. (canceled)

33. The composition of claim 1, wherein the RPE cells form a monolayer of cells on the PCL membrane.

34. The composition of claim 33, wherein microvilli on the RPE cells are evenly distributed on the cell surface.

35-37. (canceled)

38. A method for treating symptoms of a retinal disorder in a patient, comprising implanting in one or both eyes of the patient the composition of claim 1.

39. A method for treating symptoms of a retinal disorder in a patient, comprising:
implanting a porous poly(ε-caprolactone) (PCL) membrane comprising RPE cells adhered to the PCL membrane into one or both eyes of a patient suffering from a retinal disorder,
wherein the RPE cells were derived from stem cells isolated from the patient or from a donor, were induced to differentiate into RPE cells, were contacted with the PCL membrane, and were adhered to the membrane.

40. The method of claim 39, wherein the retinal disorder is age-related macular degeneration or retinitis pigmentosa.

41. The method of claim 39, wherein the retinal disorder comprises choroidal new vessel growth (CNV), atrophy of the fovea, atrophy of the subfoveal retinal pigment epithelium, atrophy of the choroid, and/or loss of central vision.

42-50. (canceled)

51. The method of claim 39, wherein the RPE cells growing on the porous PCL restore central vision.

52. A method for manufacturing a composition comprising retinal pigment epithelium (RPE) cells and a porous poly(ε-caprolactone) (PCL) membrane, comprising contacting the RPE cells with a porous PCL membrane, whereby the RPE cells adhere to the porous PCL membrane.

53. The method of claim 52, further comprising manufacturing the porous PCL membrane by sacrificial molding, injection molding, material printing, and/or laser machining.

54-60. (canceled)

61. The method of claim 52, further comprising culturing the RPE cells on the porous PCL membrane and implanting the RPE cells and the porous PCL membrane into a patient.

62. (canceled)

63. The method of claim 52, wherein the RPE cells are human cells.


SEQUENCE LISTING (23 sequences)

SEQ ID NO: 1 - protein, 5 aa, Artificial Sequence
  source: /note=“Description of Artificial Sequence Synthetic peptide”
YIGSR

SEQ ID NO: 2 - DNA, 20 bp, Artificial Sequence
  source: /note=“Description of Artificial Sequence Synthetic primer”
cgcatcaaggagttgggaat

SEQ ID NO: 3 - DNA, 24 bp, Artificial Sequence
  source: /note=“Description of Artificial Sequence Synthetic primer”
gttttccccagttgtaatagtgcc

SEQ ID NO: 4 - DNA, 17 bp, Artificial Sequence
  source: /note=“Description of Artificial Sequence Synthetic primer”
taagcaaccgccttacg

SEQ ID NO: 5 - DNA, 17 bp, Artificial Sequence
  source: /note=“Description of Artificial Sequence Synthetic primer”
agccatgcagtccgaat

SEQ ID NO: 6 - DNA, 22 bp, Artificial Sequence
  source: /note=“Description of Artificial Sequence Synthetic primer”
gctgctggagaatgaggaaact

SEQ ID NO: 7 - DNA, 23 bp, Artificial Sequence
  source: /note=“Description of Artificial Sequence Synthetic primer”
acagccttcttcgtcagtatcgt

SEQ ID NO: 8 - DNA, 21 bp, Artificial Sequence
  source: /note=“Description of Artificial Sequence Synthetic primer”
gggcagaatcatcacgaagtg

SEQ ID NO: 9 - DNA, 24 bp, Artificial Sequence
  source: /note=“Description of Artificial Sequence Synthetic primer”
tatcaccttaaccagcctttcatc

SEQ ID NO: 10 - DNA, 20 bp, Artificial Sequence
  source: /note=“Description of Artificial Sequence Synthetic primer”
gcgacccacacgtcaaacta

SEQ ID NO: 11 - DNA, 21 bp, Artificial Sequence
  source: /note=“Description of Artificial Sequence Synthetic primer”
tcagtcaacgggggacataaa

SEQ ID NO: 12 - DNA, 20 bp, Artificial Sequence
  source: /note=“Description of Artificial Sequence Synthetic primer”
cccatcaccatcttccagga

SEQ ID NO: 13 - DNA, 17 bp, Artificial Sequence
  source: /note=“Description of Artificial Sequence Synthetic primer”
ctccaggcggcgagagt

SEQ ID NO: 14 - DNA, 22 bp, Artificial Sequence
  source: /note=“Description of Artificial Sequence Synthetic primer”
tccgtaattcaatcagtcctgc

SEQ ID NO: 15 - DNA, 18 bp, Artificial Sequence
  source: /note=“Description of Artificial Sequence Synthetic primer”
gcacttagctcttcgatt

SEQ ID NO: 16 - DNA, 17 bp, Artificial Sequence
  source: /note=“Description of Artificial Sequence Synthetic primer”
actgctgctcttcagcg

SEQ ID NO: 17 - DNA, 22 bp, Artificial Sequence
  source: /note=“Description of Artificial Sequence Synthetic primer”
tgaaccgggctgggaaggaatc

SEQ ID NO: 18 - DNA, 23 bp, Artificial Sequence
  source: /note=“Description of Artificial Sequence Synthetic primer”
cgaattcctcctggtcttacaga

SEQ ID NO: 19 - DNA, 20 bp, Artificial Sequence
  source: /note=“Description of Artificial Sequence Synthetic primer”
attggatggcagtagctgcg

SEQ ID NO: 20 - DNA, 20 bp, Artificial Sequence
  source: /note=“Description of Artificial Sequence Synthetic primer”
gggtccagaatcttgcaatg

SEQ ID NO: 21 - DNA, 23 bp, Artificial Sequence
  source: /note=“Description of Artificial Sequence Synthetic primer”
tcccttgatagacacaactcctc

SEQ ID NO: 22 - DNA, 21 bp, Artificial Sequence
  source: /note=“Description of Artificial Sequence Synthetic primer”
ggggctgtactgcttaaccag

SEQ ID NO: 23 - DNA, 20 bp, Artificial Sequence
  source: /note=“Description of Artificial Sequence Synthetic primer”
catcgccccacttgattttg